(19) United States
(12) Patent Application Publication
(10) Pub. No.: US 2010/0120111 A1
(43) Pub. Date: May 13, 2010

(54) METHOD OF PRODUCING HYDROCARBON BIOFUELS USING GENETICALLY MODIFIED SEAWEED

(76) Inventor: Robert Petcavich, The Woodlands, TX (US)

Correspondence Address: GREER, BURNS & CRAIN, 300 S WACKER DR, 25TH FLOOR, CHICAGO, IL, 60606, US

(21) Appl. No.: 12/617,767

(22) Filed: Nov. 13, 2009

Related U.S. Application Data
(60) Provisional application No. 61/199,056, filed on Nov. 13, 2008.

(51) Int. Cl. C12P 5/00

(52) U.S. Cl. 435/166

(57) ABSTRACT

A method of producing hydrocarbon biofuels using genetically modified seaweed by inserting genes from high hydrocarbon producing micro algae into high growth seaweed species. In an embodiment of the present invention, Botryococcus braunii (BB) a green micro algae that produces large amounts of aliphatic hydrocarbon molecules is used as a source for genetic material. The genes for hydrocarbon production in BB are identified, removed, cloned and subsequently inserted into high growth brown seaweeds such as Kelp and hydrocarbons subsequently expressed in the plant. The genetically modified kelp is grown in coastal ocean waters, harvested, the hydrocarbons produced extracted, and catalytically converted into useful biofuels.

This application claims the benefit of U.S. Provisional Application Ser. No. 61/199,059, filed Nov. 13, 2008, under 35 U.S.C. §119.

### FIELD OF THE INVENTION

The present invention relates to hydrocarbon production by marine aquatic plant life. In particular, genetically modified fast growing seaweed species, such as kelp, are used for the production of hydrocarbons. More particularly, fast growing seaweed species are genetically modified to produce hydrocarbons that can subsequently be used as biofuels, including for transportation vehicles and chemical industry feedstocks.

### BACKGROUND OF THE INVENTION

Photosynthetic light-driven biological processes have enormous capacity for sustainable, carbon neutral, solar powered replacement of fossil fuels. Typically such fuels fix more than 100 Giga tons (Gtons) of carbon annually, which is equivalent to 100 terawatts (TW) of energy. However, this fixation rate is currently in balance with respiration and other facets of the global carbon cycle. Accordingly, adding another 10 TW of fixation would require enormous land areas at present. Agriculturally productive land is just one problem with this approach. Land based plant production also requires a tremendous amount of nitrogen fertilizer which represents up to 25% of the cost of biomass production. Worldwide, approximately 160 million tons of ammonia is produced annually by an energetically expensive fossil fuel dependent process which is or may not be sustainable for a long period of time.

Photosynthesis provides greater than 90% of the net energy into the biosphere. It produces the oxygen we breathe and drives the biogeochemical cycles. The primary reactions of photosynthesis can operate at near perfect quantum efficiency. It is therefore desirable to utilize the photosynthesis process in the harvesting of useful energy.

One goal of the present invention is to link photosynthesis and micro alga metabolism, to produce liquid hydrocarbons and useful chemical products. This is accomplished by genetically engineering into fast growing seaweed, algae genes for hydrocarbon production. While the benefits of extracting fuel hydrocarbons from algae are great, the process in the past has encountered significant difficulties. See Mayfield, Genetic Engineering of Algae for Biofuel Production, Department of Cell Biology and the Skaggs Institute for Chemical Biology, incorporated by reference herein.

In the present invention, a species of algae, Botryococcus braunii, has been reported to produce liquid hydrocarbons that represent up to 75% of the dry mass cell weight. Botryococcus braunii, is a green colonial micro algae, is an unusually rich renewable resource of hydrocarbons and other chemicals. One of the most interesting facets of the hydrocarbon production of this species is that the hydrocarbons produced are in the carbon (C25 to C40) chain length that makes them ideal candidates for catalytic conversion into fuels for transportation vehicles and chemical feed stocks. By using aquatic marine sea plant life in particular algae, to generate large amounts of hydrocarbons one solves the problem of using large amounts of land area and energy intensive fertilizer to produce biofuels.

### SUMMARY OF THE INVENTION

The object of this invention is to provide a renewable resource for hydrocarbon production used as biofuels for transportation and feedstock for chemical production.

Another object of this invention is to create genetically modified fast growing seaweed that produces hydrocarbons that can be used as biofuel and molecular feedstock for chemical production.

Another object of this invention is to provide hydrocarbon producing seaweed that can be grown in marine coastal areas for easy harvesting and processing.

Yet another object of this invention is to genetically modify fast growing seaweeds for production of hydrocarbons by inserting genes from known high hydrocarbon producing micro algae into the seaweed cells.

Another object of this invention is to directly convert solar energy into hydrocarbons for use as biofuels and chemical feedstock's using genetically modified seaweeds.

### DESCRIPTION OF A PREFERRED EMBODIMENT

Unicellular photosynthetic micro alga has the ability to produce large quantities of hydrocarbons within their cells. This conversion of solar energy directly into hydrocarbons and subsequently biofuels is of great commercial interest. One micro alga Botryococcus braunii (BB) a member of the chlorophyta family is regarded as a source of renewable fuel because of its ability to produce large amounts of hydrocarbons. Depending on the strain and growth conditions, up to 75% of algal dry mass can be hydrocarbons. The chemical nature of the hydrocarbons varies with the strain of BB alga.

Three races of BB have been documented, and these can be differentiated on the basis of hydrocarbons they produce. The race A produces odd numbered carbon C25 to C31, n alkadienes, and trienes. The B race produces triterpenoid hydrocarbons known as botryococcenes (CnH2n-10, n=30-37), and the L race produces lycopadiene, a C40 tetraterpene. Although BB is the preferred hydrocarbon producing micro alga of the present invention many others can be used and a good summary of the potential candidates can be found in a report by the United States department of Energy NREL/TP-580-24190 entitled “A Look Back at the Department of Energy's Aquatic Species Program: Biodiesel from Algae,” incorporated herein by reference.

The genetic manipulation of algae species to produce a new hybrid strain is well known in the art. For example, the hybridization of two species of algae through growing the species in close proximity in a nutrient solution is shown in U.S. Pat. No. 5,365,018 as well as U.S. Pat. No. 5,585,544, both titled “Method of Causing Somatic Hybridization Between Two Species of Algae,” and both of which are incorporated by reference herein. Similarly, the introduction of foreign DNA into algae spore protoplasts to form a hybrid fusion is shown in U.S. Pat. No. 5,426,040 “Methods for Producing Improved Sharing of Seaweed by Fusion of Spore-Protoplasts, and Resultant Seaweeds and Phycocolloids.”

The genetic material responsible for the production of the various hydrocarbons of BB is isolated, purified, and cloned into the particular organism of interest by techniques well known in the art of molecular biology. See, for example, Cheney, D. P. 1990, Genetic Improvement of Seaweeds Through Protoplast Fusion, Economically Important Marine Plants of the Atlantic: Their Biology and Cultivation, C. Yarish and C. Penniman, (eds.) Univ. of Conn. Sea Grant Program, pp. 15-25; Cheney, D., et al., 1998, Genetic Manipulation and Strain Improvement in Commercially Valuable Red Seaweeds, New Developments in Marine Biotechnology, Y. L. Gal and H. Halvorson (eds.), Plenum Press, NY, pp. 101-104; Cheney, D. P., 1999, Strain Improvement of Seaweeds Thru Genetic Manipulation: Current Status, World Aquaculture, 30: 55-56 & 65; Qin, S., Tong, S., Zhang, P. et al., 1993, Isolation of Plasmid from the Blue-green Alga, Spirulina platensis, Chinese Journal of Oceanology and Limnology 11(3): 285-288; Qin, S., Jiang, P., Li, X. et al., 1988, The Expression of lacZ in Regenerated Sporophytes of Parthenogenetic laminaria japonica, Proceedings of the 2nd Asia-Pacific Marine Biotechnology Conference and 3rd Asia-Pacific Conference on Algal Biotechnology (in press); Qin, S., Wu, J., Wang, X. et al., 1997, Expression of Foreign Genes in Laminaria japonica, The Marine Biology of the South China Sea, Morton, B. ed. Hong Kong University Press, Hong Kong, p. 3-11, all of which are incorporated by reference herein.

In the present invention the organism of interest is seaweed and in particular the pacific giant kelp. Giant kelp is a species of marine alga found on the pacific coast of North and Central America. It begins life as a microscopic spore but in many species they grow up to 60 meters (270 feet) with the upper fronds forming a dense canopy at the surface. Studies suggest kelp fronds may grow at 1-2 feet per day which makes it an ideal species to produce large amounts of hydrocarbons in a very short period of time. The giant kelp is a perennial plant that is a renewable resource for hydrocarbon production and biofuel generation. See, for example, Copping, et al, 2008, Techno-Economic Feasibility Analysis of Offshore Seaweed Farming for Bioenergy and Biobased products, Independent Research and Development Report, Battelle Pacific Northwest Division, incorporated by reference herein. The fronds are harvested and subsequently processed to extract the hydrocarbons and other chemicals of interest. Although the pacific giant kelp is preferred in the present invention many other species of seaweed can be used that are indigenous to various geographic regions throughout the world to maximize hydrocarbon production. Extraction of the hydrocarbons produced by the seaweed can be accomplished by several methodologies such as pressing and solvent extraction. Solvent extraction is the preferred method of recovery in the present invention. Under suitable condition 70% of hydrocarbons can be released by 30 minutes of contact with hexane. Hydrocarbons can be continuously extracted with proper agitation and recovery methodologies known in the prior art. Supercritical fluid (SCF) extraction can also be applied. Fluids such as carbon dioxide (CO2) allow rapid extraction is nontoxic, inexpensive, easily removed from extract and reusable.

Hydrocarbons obtained in hexane extraction of BB modified seaweed can be directly combusted, however for performance in internal combustion engines, the oil must be modified by processes such as pyrolysis and catalytic cracking. Crude hydrocarbons of BB genetically modified seaweed can be converted to gasoline, light cycle oil, heavy cycle oil, and coke by subjecting the extract to catalytic cracking as has been taught by Kitazato et al., Sekiyu Gakkaishi 32:28. The yields of gasoline obtained by catalytic cracking of algal hydrocarbons are comparable to yields obtained from petroleum. Also the gasoline produced has sufficiently high octane numbers for direct use in transportation vehicles.

The invention herein is described by examples and a particularly desired way of practicing the invention has been described. However, the invention as claimed herein is not limited to that specific description in any manner. Elements and features described in conjunction with a particular embodiment are not limited to use therewith and may be used separately or in conjunction with the other embodiments disclosed herein. Equivalence to the description as hereinafter claimed is considered to be within the scope of protection of this patent such as the use micro alga hydrocarbon producing genes cloned into to land based plant life to generate hydrocarbons for biofuels such as fast growing bamboo plants.

What is claimed is:

1. A method of producing a liquid hydrocarbon biofuel comprising:
identifying genetic material in macroalgae Botryococcus braunii which encodes cells to produce hydrocarbons;
isolating and purifying said genetic material from the micro algae;
cloning said genetic material into a designated macroalgae seaweed species to create genetically modified seaweed;
extracting hydrocarbons generated by said genetically modified seaweed through photosynthesis; and
processing said extracted hydrocarbons as liquid biofuel.

2. The method of claim 1 wherein said macroalgae seaweed species is Macrocystis pyrifera.

3. The method of claim 1 wherein said hydrocarbons are extracted through supercritical fluid extraction.

4. The method of claim 1 wherein said hydrocarbons are extracted with liquid hydrocarbon solvents.

5. The method of claim 4 wherein said liquid hydrocarbon solvents are hexane.

6. The method of claim 1 wherein said hydrocarbons are extracted through high pressure pressing.

7. The method of claim 1 wherein said processing is achieved by catalytic cracking.

8. The method of claim 1 wherein said processing is achieved by pyrolysis.

9. The method of claim 1 wherein said hydrocarbons have carbon chain lengths in the range of 5 to 60 carbons.

10. The method of claim 9 wherein said carbon chain length is in the range of 20-45 carbons.

11. The method of claim 1 further including subjecting said liquid biofuel to catalytic conversion.

12. The method of claim 11 wherein the catalytically converted biofuel is suitable for use in transportation vehicles.

13. The method of claim 11 wherein the catalytically converted biofuel is suitable for use in chemical feed stock.

14. A method of producing a liquid hydrocarbon biofuel comprising:
identifying genetic material in a strain of the macroalgae Botryococcus braunii, which encodes cells to produce hydrocarbons;
isolating and purifying said genetic material from the macroalgae;
cloning said genetic material into a species of Macrocystis pyrifera to produce modified seaweed;
extracting hydrocarbons generated by said modified seaweed through photosynthesis; and
processing said extracted hydrocarbons as liquid biofuel.

15. The method of claim 10 wherein said strain of Botryococcus braunii is an A strain.

16. The method of claim 14 wherein said strain of Botryococcus braunii is a B strain.

17. The method of claim 14 wherein said strain of Botryococcus braunii is an L strain.

18. The method of claim 14 wherein said liquid biofuel is suitable for use in transportation vehicles.

19. The method of claim 14 wherein said liquid biofuel is suitable for use in chemical feed stock.

20. A method of producing a liquid hydrocarbon biofuel suitable for use in transportation vehicles comprising:
identifying genetic material in a strain of the macroalgae Botryococcus braunii, which encodes cells to produce hydrocarbons;
isolating and purifying said genetic material from the micro algae;
cloning said genetic material into a species of Macrocystis pyrifera to produce modified seaweed;
extracting hydrocarbons generated by said modified seaweed through photosynthesis by solvent extraction;
processing said extracted hydrocarbons as liquid biofuel; and
catalytically converting said liquid biofuel.
